(19) United States
(12) Patent Application Publication
(10) Pub. No.: US 2023/0220502 A1
(43) Pub. Date: Jul. 13, 2023

(54) ALLULOSE SYRUP

(71) Applicant: SAVANNA INGREDIENTS GMBH, Elsdorf (DE)

(72) Inventors: Timo Johannes KOCH, Elsdorf (DE); Sebastian HANFT, Köln (DE)

(21) Appl. No.: 17/928,994

(22) PCT Filed: Jun. 4, 2021
(86) PCT No.: PCT/EP2021/065000

§ 371 Date: Dec. 1, 2022

(30) Foreign Application Priority Data
Jun. 5, 2020 (EP) 20178424.6

(51) Int. Cl. C13K 13/00; A23L 29/30; A23L 27/30

(52) U.S. Cl. CPC C13K 13/007; A23L 29/30; A23L 27/33

(57) ABSTRACT

The invention relates to a process for the preparation of an allulose syrup containing allulose at a product concentration of more than 70 wt.-%, relative to the total weight of the allulose syrup, the process comprising the steps of (a) providing an aqueous solution containing allulose at an educt concentration of at most 70 wt.-%, relative to the total weight of the solution; and (b) evaporating water at a temperature of the solution of less than 60° C. and under reduced pressure thereby increasing the concentration of allulose in the aqueous solution starting from the educt concentration until the product concentration is reached.

Priority is claimed of European patent application no. 20178424.6, filed on Jun. 5, 2020.

The invention relates to a process for the preparation of an allulose syrup containing allulose at a product concentration of more than 70 wt.-%, relative to the total weight of the allulose syrup, the process comprising the steps of (a) providing an aqueous solution containing allulose at an educt concentration of at most 70 wt.-%, relative to the total weight of the solution; and (b) evaporating water at a temperature of the solution of less than 60° C. and under reduced pressure thereby increasing the concentration of allulose in the aqueous solution starting from the educt concentration until the product concentration is reached.

Various processes for the preparation of allulose syrups are known from the prior art.

CN 109 306 365 relates to a preparation method of D-allulose, particularly to a method for preparing D-allulose through vacuum spray drying and belongs to the technical field of food processing. The method comprises the following steps: converting a D-allulose solution by using biological enzyme, decolorizing, carrying out ion exchange, concentrating and carrying out chromatographic separation to obtain high-purity D-allulose syrup, and further obtaining D-allulose powder by using a vacuum spray drying technology.

CN 110 627 847 relates to a method for preparing psicose crystals.

GB 2 536 304 relates to an allulose syrup that has a total dry solids content of from 70% to 80% by weight, and comprises allulose in an amount of at least 90% by weight on a dry solids basis. The pH of the syrup is from 3.0 to 5.0. The pH of the syrup has been found to be optimal in minimizing allulose degradation and hydroxymethylfurfural formation, whilst minimizing undesirable color formation over time.

US 2018 049458 relates to allulose syrups, use of allulose syrups in the manufacture of food or beverage products, and food and beverage products made using the allulose syrups.

US 2018 255814 relates to a mixed saccharide composition containing psicose, glucose and fructose with improved sweetness quality and crystallization, and a method for preventing crystallization of a mixed saccharide composition containing a psicose.

WO 2017 150766 relates to a method of producing D-psicose. The method of producing D-psicose includes subjecting D-fructose to D-psicose epimerization to produce a D-psicose-containing solution, subjecting the D-psicose-containing solution to first cooling and ion purification, subjecting the purified D-psicose-containing solution to first concentration and second cooling, subjecting the D-psicose-containing solution, which has been subjected to first concentration and second cooling, to chromatography to obtain a D-fructose-containing mother liquor and a D-psicose-containing separated solution, and subjecting the D-psicose-containing separated solution to second concentration and third cooling to obtain D-psicose crystals, wherein the D-fructose-containing mother liquor produced by chromatography is reused in the D-psicose epimerization.

WO 2019 083069 relates to an allulose syrup and a method for manufacturing same and, more specifically, to an allulose syrup and a method for manufacturing same, the allulose syrup comprising a viscosity controlling agent and a dispersing agent, and having an appropriate range of viscosity.

WO 2019 088654 relates to: a syrup comprising a citrus extract and saccharides including allulose; a method for manufacturing the syrup, the method comprising a step for mixing the citrus extract, the saccharides including allulose and an acidity regulator; a food composition comprising the syrup comprising the citrus extract and the saccharides including allulose; a flavor-improving composition comprising the citrus extract and the saccharides including allulose; a method for improving the flavor retention of the citrus extract, the method comprising a step for adding the saccharides including allulose to the citrus extract; and a flavor-manifesting composition comprising the citrus extract and the saccharides including allulose.

US 2019 029299 discloses a syrup composition and a food comprising the same. The syrup composition includes: gum, pectin, or a combination thereof; and allulose

US 2019 297931 relates to an aqueous liquid composition comprising allulose, wherein the weight content of allulose is at least 10 wt.-%, relative to the total weight of the liquid composition; and wherein the weight content of allulose is at least 10 wt.-%, relative to the total content of all carbohydrates that are contained in the liquid composition; and wherein the liquid composition has a viscosity of not more than 200 mPa·s.

US 2019 328014 discloses a D-allulose syrup including, besides D-allulose, a D-allulose dimer mass content, expressed in terms of dry mass, greater than 1.5%.

US 2020 085090 discloses a D-allulose syrup including, besides D-allulose, a D-allulose dimer mass content, expressed in terms of dry mass, lower than 1.5%.

The allulose syrups of the prior art are not satisfactory in every respect and there is a demand for improved allulose syrups.

It has been found that allulose in comparison to other sugars such as sucrose has a much more pronounced tendency to browning during processing and upon storage of allulose syrups. This browning is disadvantageous, because it alters the visual appearance of allulose syrups and accordingly also the visual appearance of beverages and foodstuffs that are prepared from the allulose syrups.

Further, it has been found that allulose in comparison to other sugars such as sucrose has a much more pronounced tendency to change smell, taste and organoleptic properties during processing and upon storage of allulose syrups. This change of smell, taste and organoleptic properties is disadvantageous, because it alters not only the smell, taste and organoleptic properties the of allulose syrups as such but also the corresponding properties of beverages and foodstuffs that are prepared from the allulose syrups.

Still further, it has been found that browning on the one hand and change of smell, taste and organoleptic properties on the other hand go along with one another, whereas on a molecular level the chemical entities that are formed during processing and upon storage of allulose syrups and that cause the brownish color are not necessarily identical to those chemical entities that are formed during processing and upon storage of allulose syrups and that cause the alterations of smell, taste and organoleptic properties.

It is an object of the invention to provide allulose syrups that have advantages compared to the allulose syrups of the prior art. The allulose syrups should be colorless or at least have no dominant brownish color. Further, the allulose syrups should have smell, taste and organoleptic properties of pure allulose in aqueous solutions at a comparatively high concentration of allulose without the superimposing notes that are conventionally formed during processing and upon storage. Still further, the allulose syrups should have excellent shelf-life and storage stability under ambient storage conditions as well as under accelerated (i.e. stressed) storage conditions without requiring special additives or other alterations of the properties of the allulose syrups such as pH adjustment. Furthermore, the allulose syrups should be obtainable by processes that can be performed on industrial scale in an economic and timely manner without excessive energy consumption e.g. for heating and/or evacuation.

This object has been achieved by the subject-matter of the patent claims.

It has been surprisingly found that in aqueous solutions the tendency of allulose to browning is significant at comparatively high concentrations of allulose, especially above 70 wt.-% relative to the total weight of the solutions, whereas at lower concentrations the stability of allulose against browning is higher. Therefore, at concentrations of up to 70 wt.-%, allulose solutions are relatively robust with respect to browning.

Further, it has been surprisingly found that in aqueous solutions the tendency of allulose to change smell, taste and organoleptic properties is significant at comparatively high concentrations of allulose, especially above 70 wt.-% relative to the total weight of the solutions, whereas at lower concentrations the stability of allulose against change of smell, taste and organoleptic properties is higher. Therefore, at concentrations of up to 70 wt.-%, allulose solutions are relatively robust with respect to change of smell, taste and organoleptic properties.

Still further, it has been surprisingly found that even in aqueous solutions having a comparatively high concentrations of allulose, especially above 70 wt.-% relative to the total weight of the solutions, browning on the one hand and change of smell, taste and organoleptic properties on the other hand can still be suppressed if the solutions are not heated beyond a certain threshold temperature, especially 60° C. or more.

Yet further, it has been surprisingly found that a two-stage process is suitable to provide aqueous solutions having comparatively high concentrations of allulose, especially above 70 wt.-%, relative to the total weight of the solutions, while suppressing browning on the one hand and change of smell, taste and organoleptic properties on the other hand. It has been surprisingly found that the solutions in the first stage of the process can be heated to higher temperatures, especially above 60° C., as long as the concentration of allulose is below 70 wt.-%, relative to the total weight of the solution. Further, it has been surprisingly found that in the second stage of the process browning as well as change of smell, taste and organoleptic properties can further be suppressed if the solutions are not heated beyond a certain threshold temperature, especially 60° C. or more.

FIG. 1 shows the relative change of dry substance content (DS) upon storage at various temperatures.

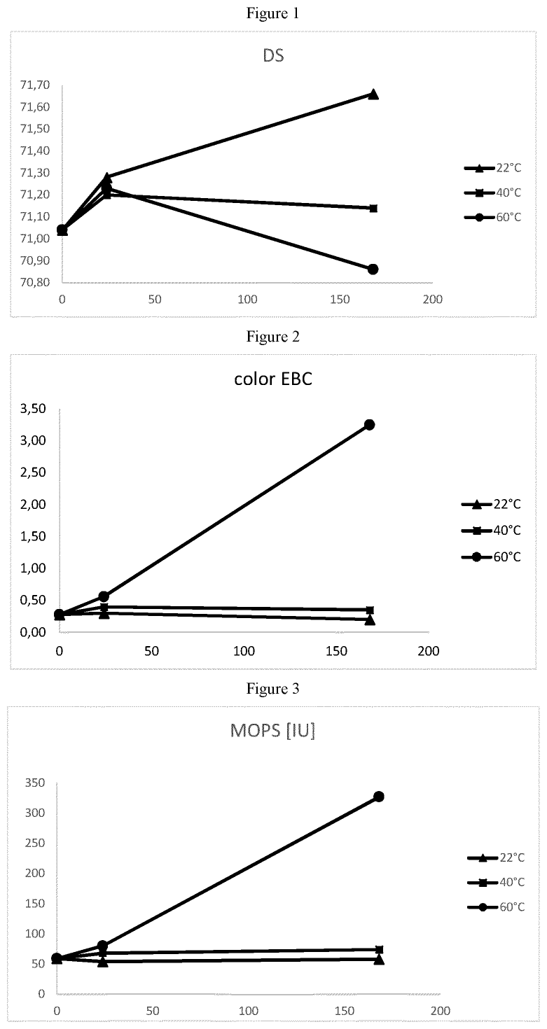

FIG. 2 shows the relative change of color (European Brewery Convention, EBC) upon storage at various temperatures.

FIG. 3 shows the relative change of color (MOPS method, ICUMSA, IU) upon storage at various temperatures.

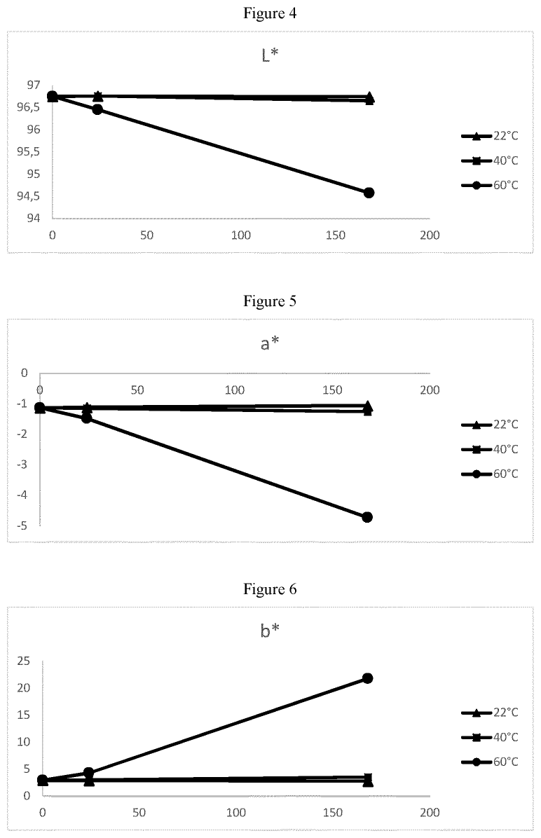

FIGS. 4 to 6 show the relative change of color in the CIELAB color space (FIG. 4 L*, FIG. 5 a*, and FIG. 6 b*) upon storage at various temperatures.

A first aspect of the invention relates to a process for the preparation of an allulose syrup containing allulose at a product concentration of more than 70 wt.-%, preferably at least 75 wt.-%, more preferably at least 77.5 wt.-%, still more preferably at least 80 wt.-%, yet more preferably at least 82.5 wt.-%, even more preferably at least 85 wt.-%, in each case relative to the total weight of the allulose syrup; the process comprising the steps of
(a) providing an aqueous solution containing allulose at an educt concentration of at most 70 wt.-%, relative to the total weight of the solution; and
(b) evaporating water at a temperature of the solution of less than 60° C. and under reduced pressure thereby increasing the concentration of allulose in the aqueous solution starting from the educt concentration until the product concentration is reached.

The process according to the invention serves the purpose of preparing of an allulose syrup comprising or essentially consisting of allulose and water.

The process according to the invention comprises at least steps (a) and (b), but may comprise additional steps prior to step (a) and/or after step (b).

For the purpose of the specification, unless expressly stated otherwise, “allulose” refers to D-allulose. While it is contemplated that D-allulose may also be present in admixture with minor amounts of L-allulose, the content of D-allulose is preferably at least 95 wt.-%, more preferably at least 99 wt. %, and in particular at least 99.9 wt.-% of the total quantity of D-allulose and L-allulose.

For the purpose of the specification, unless expressly stated otherwise, “until the educt concentration is reached” refers to the uninterrupted time span from evaporating water from the starting material containing allulose at the starting concentration until the educt concentration is reached. Thus, during this time span, the temperature of the solution is more than 35° C. and the pressure is reduced compared to atmospheric pressure.

For the purpose of the specification, unless expressly stated otherwise, “until the product concentration is reached” refers to the uninterrupted time span from evaporating water from the aqueous solution containing allulose at the educt concentration until the product concentration is reached. Thus, during this time span, the temperature of the solution is less than 60° C. and the pressure is reduced compared to atmospheric pressure.

For the purpose of the specification, unless expressly stated otherwise, “essentially consisting of” means that additional components other than those explicitly recited amount to at most 5 wt.-%, preferably at most 2.5 wt.-%, more preferably at most 1.0 wt.-%.

For the purpose of the specification, unless expressly stated otherwise, “reduced pressure” means that the pressure is reduced compared to atmospheric pressure, i.e. that a vacuum is present. Thus, “lower pressure” means that the vacuum is stronger and therefore the absolute pressure expressed in mbar decreases. “Higher pressure” accordingly means that the vacuum is weaker and therefore the absolute pressure expressed in mbar increases.

Unless expressly stated otherwise, all percentages are expressed as weight percent (wt.-%).

In step (a) of the process according to the invention, an aqueous solution is provided containing allulose at an educt concentration of at most 70 wt.-%, relative to the total weight of the solution.

While it is contemplated that the aqueous solution provided in step (a) may additionally contain undissolved material in suspension, preferably the aqueous solution is a pure solution. Preferably, the aqueous solution provided in step (a) comprises or essentially consists of allulose and water.

Preferably, the aqueous solution provided in step (a) has a density of less than 1.36 g·cm⁻³, more preferably less than 1.33 g·cm³, still more preferably less than 1.30 g·cm⁻³, yet more preferably less than 1.27 g·cm⁻³, even more preferably less than 1.24 g·cm⁻³, most preferably less than 1.21 g·cm⁻³, and in particular less than 1.18 g·cm³.

Preferably, the aqueous solution provided in step (a) contains allulose at an educt concentration of at least 50 wt.-%, preferably at least 52.5 wt.-%, more preferably at least 55 wt.-%, still more preferably at least 57.5, yet more preferably at least 60 wt.-%, relative to the total weight of the solution.

Preferably, the aqueous solution provided in step (a) contains allulose at an educt concentration of at least 50 wt.-%, preferably at least 52.5 wt.-%, more preferably at least 55 wt.-%, still more preferably at least 57.5, yet more preferably at least 60 wt.-%, even more preferably at least 62.5 wt.-%, most preferably at least 65 wt.-%, relative to the total weight of the solution.

Preferably, the aqueous solution provided in step (a) contains allulose at an educt concentration of at most 67.5 wt.-%, preferably at most 65 wt.-%, more preferably at most 62.5 wt.-%, still more preferably at most 60 wt.-%, relative to the total weight of the solution.

Preferably, the aqueous solution provided in step (a) originates from a reactor wherein allulose is synthesized from suitable starting materials, preferably from fructose, in an enzymatically catalyzed process. After synthesis of the allulose in the reactor, the product composition that has been withdrawn from the reactor may have undergone work-up, such as desalting, decoloring, purification (e.g. by chromatography), filtration (e.g. nanofiltration), preconcentration, or combinations thereof.

When the aqueous solution provided in step (a) has previously undergone preconcentration already, preferably by evaporation of water, the conditions of such preconcentration step are not particularly limited.

In especially preferred embodiments, when the aqueous solution provided in step (a) has previously undergone preconcentration already, preferably by evaporation of water, the conditions of such preconcentration step are particularly limited.

Preferably, step (a) comprises a preconcentration step, which comprises the sub-steps of
(a-1) providing a starting material containing allulose at a starting concentration of at least 25 wt.-%; and
(a-2) evaporating water at a temperature of the starting material of more than 35° C. and under reduced pressure thereby increasing the concentration of allulose in the starting material starting from the starting concentration until the educt concentration is reached and thereby providing the aqueous solution containing allulose at the educt concentration.

Preferably, step (b) is carried out after step (a).

Preferably, in step (a) of the process according to the invention, water is evaporated from the starting material provided in step (a-1) a temperature of the solution of more than 35° C. and under (reduced) pressure thereby increasing the concentration of allulose in the starting material starting from the starting concentration until the educt concentration is reached. Thus, in the course of step (a) of the process according to the invention, the starting material provided in step (a-1) is preferably converted into the aqueous solution by increasing the concentration of allulose due to evaporation of water.

For the purpose of definition, in the following “starting material” refers to any aqueous solution containing allulose at the starting concentration or above, but below the educt concentration. Once the educt concentration of allulose is reached, the starting material has been converted and the “aqueous solution” according to the invention is obtained.

Preferably, the temperature of the starting material is at most 80° C., preferably at most 78° C., more preferably at most 76° C., still more preferably at most 74° C., yet more preferably at most 72° C., even more preferably at most 70° C., most preferably at most 68° C., and in particular at most 66° C.

Preferably, the temperature of the starting material is at least 52° C., preferably at least 54° C., more preferably at least 56° C., still more preferably at least 58° C., yet more preferably at least 60° C., even more preferably at least 62° C., most preferably at least 64° C., and in particular at least 66° C.

Preferably, the temperature of the starting material is within the range of from 50 to 80° C., preferably from 52.5 to 77.5° C., more preferably from 55 to 75° C., still more preferably from 57.5 to 72.5° C., yet more preferably from 60 to 70° C., even more preferably from 62.5 to 67.5° C.

Preferably, the temperature of the starting material is kept essentially constant over time, preferably until the end of evaporation, i.e. until the educt concentration is reached; preferably the temperature of the starting material does not change relatively by more than ±2.0° C.; preferably by not more than ±1.5° C.; more preferably by not more than ±1.0° C.; most preferably by not more than ±0.5° C.

Preferably, the temperature of the starting material differs from the temperature at which step (a) is performed. For example, when the desired temperature of the starting material is reached by heating the starting material by means of a water bath, the temperature of the water bath, i.e. the temperature at which step (a) is carried out, is preferably higher than the temperature of the starting material.

Preferably, step (a) is carried out at a temperature of at most 95° C., preferably at most 90° C., more preferably at most 85° C., still more preferably at most 80° C., yet more preferably at most 75° C., even more preferably at most 70° C., most preferably at most 67° C., and in particular at most 65° C.

Preferably, step (a) is carried out at a temperature of at least 50° C., preferably at least 56° C., more preferably at least 59° C., still more preferably at least 61° C., yet more preferably at least 65° C., even more preferably at least 70° C., most preferably at least 75° C., and in particular at least 80° C.

Preferably, step (a) is carried out at a temperature within the range of from 50 to 90° C., preferably from 53 to 88° C., more preferably from 56 to 86° C., still more preferably from 59 to 84° C., yet more preferably from 61 to 82° C., even more preferably from 63 to 80° C.

Preferably, step (a) is carried out at a temperature, which is kept essentially constant over time, preferably until the end of evaporation, i.e. until the educt concentration is reached; preferably the temperature does not change relatively by more than more than ±2.0° C.; preferably by not more than ±1.5° C.; more preferably by not more than ±1.0° C.; most preferably by not more than ±0.5° C.

Preferably, step (a) is carried out at a pressure of at most 300 mbar, preferably at most 250 mbar, more preferably at most 220 mbar, still more preferably at most 190 mbar, yet more preferably at most 160 mbar, even more preferably at most 130 mbar, most preferably at most 100 mbar, and in particular at most 70 mbar.

Preferably, step (a) is carried out at a pressure of at least 50 mbar, preferably at least 70 mbar, more preferably at least 90 mbar, still more preferably at least 110 mbar, yet more preferably at least 130 mbar, even more preferably at least 150 mbar, most preferably at least 170 mbar, and in particular at least 190 mbar.

Preferably, step (a) is carried out at a pressure within the range of from 50 to 499 mbar, preferably from 70 to 399 mbar, more preferably from 100 to 350 mbar, still more preferably from 120 to 300 mbar, yet more preferably from 150 to 250 mbar.

Preferably, step (a) is carried out at a (reduced) pressure, which is kept essentially constant over time, preferably until the end of evaporation, i.e. until the educt concentration is reached; preferably the (reduced) pressure does not change relatively by more than ±20 mbar; preferably by not more than ±15 mbar; more preferably by not more than ±10 mbar; most preferably by not more than ±5 mbar.

Preferably, the starting material is an aqueous solution.

Preferably, the starting material provided in sub-step (a-1) contains allulose at a starting concentration of at least 30 wt.-%, preferably at least 35 wt.-%, more preferably at least 40 wt.-%, still more preferably at least 42 wt.-%, yet more preferably at least 44 wt.-%, even more preferably at least 46 wt. %, most preferably at least 48 wt.-%, and in particular at least 50 wt.-%, relative to the total weight of the starting material.

Preferably, the starting material provided in sub-step (a-1) contains allulose at a starting concentration of at most 69 wt.-%, preferably at most 67 wt.-%, more preferably at most 64 wt.-%, still more preferably at most 62 wt.-%, yet more preferably at most 59 wt.-%, even more preferably at most 57 wt.-%, most preferably at most 54 wt.-%, and in particular at most 52 wt.-%, relative to the total weight of the starting material.

Preferably, the starting material provided in sub-step (a-1) contains allulose at a starting concentration within the range of from 40 to 69 wt.-%, preferably from 42 to 67 wt.-%, more preferably from 44 to 65 wt.-%, still more preferably from 46 to 63 wt.-%, yet more preferably from 48 to 61 wt. %, even more preferably from 50 to 59 wt.-%, relative to the total weight of the starting material.

Preferably, the allulose content of the starting material at the starting concentration is less than the allulose content at the aqueous solution at the educt concentration.

Preferably, the starting material provided in sub-step (a-1) in the CIELAB color space has an

(i) L* value greater than 94.60; and/or
(ii) a* value greater than −4.70; and/or
(iii) b* value lower than 21.70.

Methods for determining the L* value, the a* value and the b* value according to the CIELAB color space are known to the skilled person. Color may be measured using a colorimeter, e.g. a Minolta CR-10 colorimeter. Preferably, color is measured in accordance with DIN EN ISO/CIE 11664-4:2020-03, part 4. In the CIELAB color space, L* indicates lightness and ranges from 0 (black) to 100 (white); a* indicates redness and ranges from −60 (green) to +60 (red); and b* indicates yellowness and ranges from −60 (blue) to +60 (yellow).

Preferably, the L* value of the starting material provided in step (a-1) is at least 94.70, or at least 94.80, or at least 94.90, or at least 95.00, or at least 95.10, or at least 95.20, or at least 95.30, or at least 95.40, or at least 95.50, or at least 95.60, or at least 95.70, or at least 95.80, or at least 95.90, or at least 96.00, or at least 96.10, or at least 96.20, or at least 96.30, or at least 96.40, or at least 96.50.

Preferably, the L* value of the starting material provided in step (a-1) is within the range of 96.75±2.00, more preferably 96.75±1.80, still more preferably 96.75±1.60, yet more preferably 96.75±1.40, even more preferably 96.75±1.20, most preferably 96.75±1.00, and in particular 96.75±0.80.

Preferably, the a* value of the starting material provided in step (a-1) is at least −4.50, or at least −4.00, or at least −3.50, or at least −3.00, or at least −2.50, or at least −2.00, or at least −1.90, or at least −1.80, or at least −1.70, or at least −1.60, or at least −1.50, or at least −1.45, or at least −1.40, or at least −1.35, or at least −1.30, or at least −1.25.

Preferably, the a* value of the starting material provided in step (a-1) is within the range of −1.13±4.00, more preferably −1.13±3.50, still more preferably −1.13±3.00, yet more preferably −1.13±2.50, even more preferably −1.13±2.00, most preferably −1.13±1.50, and in particular −1.13±1.00. Preferably, the a* value of the aqueous solution provided in step (a) is within the range of −1.13±0.90, more preferably −1.13±0.80, still more preferably −1.13±0.70, yet more preferably −1.13±0.60, even more preferably −1.13±0.40, most preferably −1.13±0.30, and in particular −1.13±0.20.

Preferably the b* value of the starting material provided in step (a-1) is at most 21.00, or at most 19.00, or at most 18.00, or at most 17.00, or at most 16.00, or at most 15.00, or at most 14.00, or at most 13.00, or at most 12.00, or at most 11.00, or at most 10.00, or at most 9.00, or at most 8.00, or at most 7.00, or at most 6.00, or at most 5.00, or at most 4.50, or at most 4.40, or at most 4.30, or at most 4.20, or at most 4.10, or at most 4.00, or at most 3.90, or at most 3.80, or at most 3.70, or at most 3.60, or at most 3.50.

Preferably, the b* value of the starting material provided in step (a-1) is within the range of −2.90±10.00, more preferably −2.90±9.00, still more preferably −2.90±8.00, yet more preferably −2.90±7.00, even more preferably −2.90±6.00, most preferably −2.90±5.00, and in particular −2.90±4.00. Preferably, the b* value of the aqueous solution provided in step (a) is within the range of −2.90±3.50, more preferably −2.90±3.00, still more preferably −2.90±2.50, yet more preferably −2.90±2.00, even more preferably −2.90±1.80, most preferably −2.90±1.60, and in particular −2.90±1.40.

In a preferred embodiment, the process according to the invention comprises the steps of
(a-1) providing a starting material containing allulose at a starting concentration of at least 50 wt.-%; and
(a-2) evaporating water at a temperature of the starting material of at least 62° C. and at a pressure of at most 210 mbar thereby increasing the concentration of allulose in the starting material starting from the starting concentration until the educt concentration is reached and thereby providing the aqueous solution containing allulose at the educt concentration; and
(b) evaporating water at a temperature of the solution within the range of from 46 to 54° C. and at a pressure of at most 70 mbar thereby increasing the concentration of allulose in the aqueous solution starting from the educt concentration until the product concentration is reached.

In a preferred embodiment, the conditions of the preconcentration step essentially correspond or are identical to the conditions of evaporation step (b) of the process according to the invention, i.e. the temperature of the solution and the (reduced) pressure in the preconcentration step relatively deviate from the respective conditions of evaporation step (b) by not more than 5%, preferably not more than 2%.

Thus, as according to this embodiment the conditions of the preconcentration step and the conditions of the evaporation step (b) are essentially the same, the provision of the aqueous solution in step (a) may occur in the course of an ongoing overall evaporation under these conditions, wherein an initial time period of said ongoing overall evaporation may be regarded as the preconcentration step that is performed until the educt concentration is reached, whereas the remainder of said ongoing overall evaporation may be regarded as the evaporation step (b) of the process according to the invention.

In other preferred embodiments, the conditions of the preconcentration step differ from the conditions of evaporation step (b) of the process according to the invention, (i) either in the temperature of the solution, (ii) or in the (reduced) pressure, (iii) or in the temperature of the solution and the (reduced) pressure, whereas in either case the temperature of the solution and the pressure in the preceding preconcentration step independently of one another may be higher or lower than the temperature of the solution and the pressure in subsequent evaporation step (b) of the process according to the invention.

Thus, as according to these embodiments the conditions of the preconcentration step and the conditions of the evaporation step (b) differ in at least one parameter, the provision of the aqueous solution in step (a) takes place at the end of a preceding preconcentration step that is performed until the educt concentration is reached, followed by the evaporation step (b) of the process according to the invention. For example, when the overall evaporation is performed in two different steps as preconcentration and evaporation step (b), it has been found to be energetically advantageous to omit the temperature range of between 59° C. and 71° C., i.e. to operate the evaporators at solution temperatures above and below, respectively.

In a preferred embodiment, the preconcentration step and the evaporation step (b) of the process according to the invention are performed under essentially the same (reduced) pressure, i.e. the (reduced) pressure in the preconcentration step relatively deviates from the (reduced) pressure in the evaporation step (b) by not more than 5%, preferably not more than 2%; but the temperature of the solution in the preconcentration step is lower than the temperature of the solution in the evaporation step (b) of the process according to the invention. According to this preferred embodiment, the relative difference of the temperature of the solution in the preconcentration step and the temperature of the solution in the evaporation step (b) of the process according to the invention is at least 5° C., or at least 10° C., or at least 15° C., or at least 20° C., or at least 25° C., or at least 30° C., or at least 35° C., or at least 40° C.

In another preferred embodiment, the preconcentration step and the evaporation step (b) of the process according to the invention are performed under essentially the same (reduced) pressure, i.e. the (reduced) pressure in the preconcentration step relatively deviates from the (reduced) pressure in the evaporation step (b) by not more than 5%, preferably not more than 2%; but the temperature of the solution in the preconcentration step is higher than the temperature of the solution in the evaporation step (b) of the process according to the invention. According to this preferred embodiment, the relative difference of the temperature of the solution in the preconcentration step and the temperature of the solution in the evaporation step (b) of the process according to the invention is at least 5° C., or at least 10° C., or at least 15° C., or at least 20° C., or at least 25° C., or at least 30° C., or at least 35° C., or at least 40° C.

In still another preferred embodiment, the preconcentration step and the evaporation step (b) of the process according to the invention are performed under essentially the same temperature of the solution, i.e. the temperature of the solution in the preconcentration step relatively deviates from the temperature of the solution in the evaporation step (b) by not more than 5%, preferably not more than 2%; but the (reduced) pressure in the preconcentration step is lower than the (reduced) pressure in the evaporation step (b) of the process according to the invention. According to this preferred embodiment, the relative difference of the (reduced) pressure in the preconcentration step and the (reduced) pressure in the evaporation step (b) of the process according to the invention is at least 20 mbar, or at least 40 mbar, or at least 60 mbar, or at least 80 mbar, or at least 100 mbar, or at least 120 mbar, or at least 140 mbar, or at least 160 mbar, or at least 180 mbar, or at least 200 mbar.

In a further preferred embodiment, the preconcentration step and the evaporation step (b) of the process according to the invention are performed under essentially the same temperature of the solution, i.e. the temperature of the solution in the preconcentration step relatively deviates from the temperature of the solution in the evaporation step (b) by not more than 5%, preferably not more than 2%; but the (reduced) pressure in the preconcentration step is higher than the (reduced) pressure in the evaporation step (b) of the process according to the invention. According to this preferred embodiment, the relative difference of the (reduced) pressure in the preconcentration step and the (reduced) pressure in the evaporation step (b) of the process according to the invention is at least 20 mbar, or at least 40 mbar, or at least 60 mbar, or at least 80 mbar, or at least 100 mbar, or at least 120 mbar, or at least 140 mbar, or at least 160 mbar, or at least 180 mbar, or at least 200 mbar.

In a yet further preferred embodiment, the temperature of the solution in the preconcentration step is higher than the temperature of the solution in the evaporation step (b) of the process according to the invention; and the (reduced) pressure in the preconcentration step is higher than the (reduced) pressure in the evaporation step (b) of the process according to the invention. According to this preferred embodiment, the relative difference of the temperature of the solution in the preconcentration step and the temperature of the solution in the evaporation step (b) of the process according to the invention is at least 5° C., or at least 10° C., or at least 15° C., or at least 20° C., or at least 25° C., or at least 30° C., or at least 35° C., or at least 40° C. According to this preferred embodiment, the relative difference of the (reduced) pressure in the preconcentration step and the (reduced) pressure in the evaporation step (b) of the process according to the invention is at least 20 mbar, or at least 40 mbar, or at least 60 mbar, or at least 80 mbar, or at least 100 mbar, or at least 120 mbar, or at least 140 mbar, or at least 160 mbar, or at least 180 mbar, or at least 200 mbar.

In yet another preferred embodiment, the temperature of the solution in the preconcentration step is lower than the temperature of the solution in the evaporation step (b) of the process according to the invention; and the (reduced) pressure in the preconcentration step is higher than the (reduced) pressure in the evaporation step (b) of the process according to the invention. According to this preferred embodiment, the relative difference of the temperature of the solution in the preconcentration step and the temperature of the solution in the evaporation step (b) of the process according to the invention is at least 5° C., or at least 10° C., or at least 15° C., or at least 20° C., or at least 25° C., or at least 30° C., or at least 35° C., or at least 40° C. According to this preferred embodiment, the relative difference of the (reduced) pressure in the preconcentration step and the (reduced) pressure in the evaporation step (b) of the process according to the invention is at least 20 mbar, or at least 40 mbar, or at least 60 mbar, or at least 80 mbar, or at least 100 mbar, or at least 120 mbar, or at least 140 mbar, or at least 160 mbar, or at least 180 mbar, or at least 200 mbar.

In another preferred embodiment, the temperature of the solution in the preconcentration step is higher than the temperature of the solution in the evaporation step (b) of the process according to the invention; and the (reduced) pressure in the preconcentration step is lower than the (reduced) pressure in the evaporation step (b) of the process according to the invention. According to this preferred embodiment, the relative difference of the temperature of the solution in the preconcentration step and the temperature of the solution in the evaporation step (b) of the process according to the invention is at least 5° C., or at least 10° C., or at least 15° C., or at least 20° C., or at least 25° C., or at least 30° C., or at least 35° C., or at least 40° C. According to this preferred embodiment, the relative difference of the (reduced) pressure in the preconcentration step and the (reduced) pressure in the evaporation step (b) of the process according to the invention is at least 20 mbar, or at least 40 mbar, or at least 60 mbar, or at least 80 mbar, or at least 100 mbar, or at least 120 mbar, or at least 140 mbar, or at least 160 mbar, or at least 180 mbar, or at least 200 mbar.

In a further preferred embodiment, the temperature of the solution in the preconcentration step is lower than the temperature of the solution in the evaporation step (b) of the process according to the invention; and the (reduced) pressure in the preconcentration step is lower than the (reduced) pressure in the evaporation step (b) of the process according to the invention. According to this preferred embodiment, the relative difference of the temperature of the solution in the preconcentration step and the temperature of the solution in the evaporation step (b) of the process according to the invention is at least 5° C., or at least 10° C., or at least 15° C., or at least 20° C., or at least 25° C., or at least 30° C., or at least 35° C., or at least 40° C. According to this preferred embodiment, the relative difference of the (reduced) pressure in the preconcentration step and the (reduced) pressure in the evaporation step (b) of the process according to the invention is at least 20 mbar, or at least 40 mbar, or at least 60 mbar, or at least 80 mbar, or at least 100 mbar, or at least 120 mbar, or at least 140 mbar, or at least 160 mbar, or at least 180 mbar, or at least 200 mbar.

Preferably, the aqueous solution provided in step (a) essentially consists of (i) allulose, (ii) residual by-products obtained in the course of allulose synthesis and not removed by purification, (iii) residual starting materials not converted in the course of allulose synthesis and not removed by purification, and (iv) water. Preferably, the aqueous solution provided in step (a) essentially contains no liquids (solvents) other than water.

Preferably, the aqueous solution provided in step (a) has an allulose content of at least 90 wt. %; preferably at least 95 wt.-%; more preferably at least 98 wt.-%; still more preferably at least 99 wt. %; in each case relative to the total content of dry matter that is contained in the aqueous solution.

Preferably, the aqueous solution provided in step (a) has a fructose content of at most 10 wt.-%; preferably at most 5.0 wt.-%; more preferably at most 2.5 wt.-%; in each case relative to the total content of dry matter that is contained in the aqueous solution.

Preferably, the aqueous solution provided in step (a) has a content of components other than allulose (i.e. total impurities) of at most 10 wt.-%; preferably at most 5.0 wt.-%; more preferably at most 2.5 wt.-%; in each case relative to the total content of dry matter that is contained in the aqueous solution.

Preferably, the aqueous solution provided in step (a) in the CIELAB color space has an

(i) L* value greater than 94.60; and/or
(ii) a* value greater than −4.70; and/or
(iii) b* value lower than 21.70.

Preferably, the L* value of the aqueous solution provided in step (a) is at least 94.70, or at least 94.80, or at least 94.90, or at least 95.00, or at least 95.10, or at least 95.20, or at least 95.30, or at least 95.40, or at least 95.50, or at least 95.60, or at least 95.70, or at least 95.80, or at least 95.90, or at least 96.00, or at least 96.10, or at least 96.20, or at least 96.30, or at least 96.40, or at least 96.50.

Preferably, the L* value of the aqueous solution provided in step (a) is within the range of 96.75±2.00, more preferably 96.75±1.80, still more preferably 96.75±1.60, yet more preferably 96.75±1.40, even more preferably 96.75±1.20, most preferably 96.75±1.00, and in particular 96.75±0.80.

Preferably, the a* value of the aqueous solution provided in step (a) is at least −4.50, or at least −4.00, or at least −3.50, or at least −3.00, or at least −2.50, or at least −2.00, or at least −1.90, or at least −1.80, or at least −1.70, or at least −1.60, or at least −1.50, or at least −1.45, or at least −1.40, or at least −1.35, or at least −1.30, or at least −1.25.

Preferably, the a* value of the aqueous solution provided in step (a) is within the range of −1.13±4.00, more preferably −1.13±3.50, still more preferably −1.13±3.00, yet more preferably −1.13±2.50, even more preferably −1.13±2.00, most preferably −1.13±1.50, and in particular −1.13±1.00. Preferably, the a* value of the aqueous solution provided in step (a) is within the range of −1.13±0.90, more preferably −1.13±0.80, still more preferably −1.13±0.70, yet more preferably −1.13±0.60, even more preferably −1.13±0.40, most preferably −1.13±0.30, and in particular −1.13±0.20.

Preferably the b* value of the aqueous solution provided in step (a) is at most 21.00, or at most 19.00, or at most 18.00, or at most 17.00, or at most 16.00, or at most 15.00, or at most 14.00, or at most 13.00, or at most 12.00, or at most 11.00, or at most 10.00, or at most 9.00, or at most 8.00, or at most 7.00, or at most 6.00, or at most 5.00, or at most 4.50, or at most 4.40, or at most 4.30, or at most 4.20, or at most 4.10, or at most 4.00, or at most 3.90, or at most 3.80, or at most 3.70, or at most 3.60, or at most 3.50.

Preferably, the b* value of the aqueous solution provided in step (a) is within the range of −2.90±10.00, more preferably −2.90±9.00, still more preferably −2.90±8.00, yet more preferably −2.90±7.00, even more preferably −2.90±6.00, most preferably −2.90±5.00, and in particular −2.90±4.00. Preferably, the b* value of the aqueous solution provided in step (a) is within the range of −2.90±3.50, more preferably −2.90±3.00, still more preferably −2.90±2.50, yet more preferably −2.90±2.00, even more preferably −2.90±1.80, most preferably −2.90±1.60, and in particular −2.90±1.40.

In step (b) of the process according to the invention, water is evaporated from the aqueous solution provided in step (a) a temperature of the solution of less than 60° C. and under (reduced) pressure thereby increasing the concentration of allulose in the aqueous solution starting from the educt concentration until the product concentration is reached. Thus, in the course of step (b) of the process according to the invention, the aqueous solution provided in step (a) is converted into the allulose syrup by increasing the concentration of allulose due to evaporation of water.

For the purpose of definition, in the following “aqueous solution” refers to any aqueous solution containing allulose at the educt concentration or above, but below the product concentration. Once the product concentration of allulose is reached, the aqueous solution has been converted and “allulose syrup” according to the invention is obtained.

Preferably, the temperature of the solution is at most 55° C., preferably at most 50° C., more preferably at most 45° C., still more preferably at most 40° C., yet more preferably at most 37° C.

Preferably, the temperature of the solution is at most 58° C., preferably at most 56° C., more preferably at most 54° C., still more preferably at most 52° C., yet more preferably at most 50° C., even more preferably at most 48° C., most preferably at most 46° C., and in particular at most 44° C.

Preferably, the temperature of the solution is at least 36° C., preferably at least 38° C., more preferably at least 40° C., still more preferably at least 42° C., yet more preferably at least 44° C., even more preferably at least 46° C., most preferably at least 48° C., and in particular at least 50° C.

Preferably, the temperature of the solution is kept essentially constant over time, preferably until the end of evaporation, i.e. until the product concentration is reached; preferably the temperature of the solution does not change relatively by more than ±2.0° C.; preferably by not more than ±1.5° C.; more preferably by not more than ±1.0° C.; most preferably by not more than ±0.5° C.

Preferably, the temperature of the solution differs from the temperature at which step (b) is performed. For example, when the desired temperature of the solution is reached by heating the solution by means of a water bath, the temperature of the water bath, i.e. the temperature at which step (b) is carried out, is preferably higher than the temperature of the solution.

Preferably, step (b) is carried out at a temperature of at most 70° C., preferably at most 65° C., more preferably at most 63° C., still more preferably at most 61° C., yet more preferably at most 59° C., even more preferably at most 57° C., most preferably at most 56° C., and in particular at most 54° C.

Preferably, step (b) is carried out at a temperature of at least 36° C., preferably at least 38° C., more preferably at least 40° C., still more preferably at least 42° C., yet more preferably at least 44° C., even more preferably at least 46° C., most preferably at least 48° C., and in particular at least 50° C.

Preferably, step (b) is carried out at a temperature within the range of from 36 to 70° C., preferably from 38 to 65° C., more preferably from 40 to 60° C., still more preferably from 42 to 58° C., yet more preferably from 44 to 56° C., even more preferably from 46 to 54° C.

Preferably, step (b) is carried out at a temperature, which is kept essentially constant over time, preferably until the end of evaporation, i.e. until the product concentration is reached; preferably the temperature does not change relatively by more than more than ±2.0° C.; preferably by not more than ±1.5° C.; more preferably by not more than ±1.0° C.; most preferably by not more than ±0.5° C.

Preferably, step (b) of the process according to the invention additionally includes maintaining, preferably until the end of evaporation, i.e. until the product concentration is reached, the vapor phase above the aqueous solution at a vapor temperature within the range of from 25 to 65° C.; preferably 29 to 60° C.

Preferably, the vapor temperature is at least 25° C., or at least 27° C., or at least 29° C., or at least 31° C., or at least 33° C., or at least 35° C., or at least 37° C., or at least 39° C., or at least 41° C., or at least 43° C., or at least 45° C., or at least 47° C., or at least 49° C., or at least 51° C., or at least 53° C., or at least 55° C.

Preferably, the vapor temperature is at most 65° C., or at most 63° C., or at most 61° C., or at most 59° C., or at most 57° C., at most 55° C., or at most 53° C., or at most 51° C., or at most 49° C., or at most 47° C., or at most 45° C., or at most 43° C., or at most 41° C., or at most 39° C., or at most 37° C., or at most 35° C., or at most 33° C., or at most 31° C., or at most 29° C., or at most 27° C., or at most 25° C.

Preferably, the vapor phase above the aqueous solution is kept over time at an essentially constant vapor temperature; preferably the vapor temperature does not change relatively by more than ±2.0° C.; preferably by not more than ±1.5° C.; more preferably by not more than ±1.0° C.; most preferably by not more than ±0.5° C.

Preferably, the pressure is at most 500 mbar, preferably at most 200 mbar, more preferably at most 100 mbar, still more preferably at most 80 mbar.

Preferably, the pressure is at least 50 mbar, preferably at least 70 mbar, more preferably at least 90 mbar, still more preferably at least 110 mbar, yet more preferably at least 130 mbar, even more preferably at least 150 mbar, most preferably at least 170 mbar, and in particular at least 190 mbar.

Preferably, the (reduced) pressure is kept essentially constant over time, preferably until the end of evaporation, i.e. until the product concentration is reached; preferably the (reduced) pressure does not change relatively by more than ±20 mbar; preferably by not more than ±15 mbar; more preferably by not more than ±10 mbar; most preferably by not more than ±5 mbar.

Preferably, step (b) is carried out at a pressure of at most 499 mbar, preferably at most 399 mbar, more preferably at most 350 mbar, still more preferably at most 300 mbar, yet more preferably at most 250 mbar, even more preferably at most 200 mbar, most preferably at most 150 mbar, and in particular at most 100 mbar.

Preferably, step (b) is carried out at a pressure of at least 50 mbar, preferably at least 70 mbar, more preferably at least 90 mbar, still more preferably at least 110 mbar, yet more preferably at least 130 mbar, even more preferably at least 150 mbar, most preferably at least 170 mbar, and in particular at least 190 mbar.

Preferably, step (b) is carried out at a pressure within the range of from 50 to 499 mbar, preferably from 70 to 399 mbar, more preferably from 90 to 300 mbar, still more preferably from 110 to 250 mbar, yet more preferably from 130 to 220 mbar.

Preferably, step (b) is carried out at a (reduced) pressure, which is kept essentially constant over time, preferably until the end of evaporation, i.e. until the product concentration is reached; preferably the (reduced) pressure does not change relatively by more than ±20 mbar; preferably by not more than ±15 mbar; more preferably by not more than ±10 mbar; most preferably by not more than ±5 mbar.

It has been found that allulose is particularly prone to color formation especially at comparatively high temperatures of the solution and at comparatively high concentrations of allulose in the solution. Color formation is a function of time. Thus, the longer the allulose solution is subjected to elevated temperature, the more color will be formed. Thus, when evaporating water from the allulose solution at elevated temperature, care should be taken that evaporation proceeds quickly so that the allulose is not subjected too long to the elevated temperature in order to avoid color formation.

It has been found that with respect to color formation shorter evaporation times cannot be compensated by higher temperatures. Thus, although less time is needed for evaporation of water when the temperature of the solution is increased so that the allulose solution is subjected to the increased temperature for a shorter period of time, nonetheless significant amounts of colored by-products are formed.

Under (reduced) pressure, evaporation temperatures can be kept considerably low. While at the same time less energy is needed for heating, more energy is required for maintaining the (reduced) pressure.

It has been found that energy consumption, time of evaporation and suppression of color formation can be optimized by properly adjusting temperature of the solution and pressure at the given concentration of allulose in solution.

Preferred temperatures of the solution T, preferred pressures p and preferred educt concentrations c are compiled as embodiments A¹ to A⁸⁰ in the table here below:

| | A¹ | A² | A³ | A⁴ | A⁵ | A⁶ | A⁷ | A⁸ | A⁹ | A¹⁰ |
|---|---|---|---|---|---|---|---|---|---|---|
| T [° C.] | ≤55 | ≤50 | ≤45 | ≤40 | ≤37 | ≤55 | ≤50 | ≤45 | ≤40 | ≤37 |
| p [mbar] | ≤500 | ≤500 | ≤500 | ≤500 | ≤500 | ≤200 | ≤200 | ≤200 | ≤200 | ≤200 |
| c [wt.-%] | ≥50 | ≥50 | ≥50 | ≥50 | ≥50 | ≥50 | ≥50 | ≥50 | ≥50 | ≥50 |
| | A¹¹ | A¹² | A¹³ | A¹⁴ | A¹⁵ | A¹⁶ | A¹⁷ | A¹⁸ | A¹⁹ | A²⁰ |
| T [° C.] | ≤55 | ≤50 | ≤45 | ≤40 | ≤37 | ≤55 | ≤50 | ≤45 | ≤40 | ≤37 |
| p [mbar] | ≤100 | ≤100 | ≤100 | ≤100 | ≤100 | ≤80 | ≤80 | ≤80 | ≤80 | ≤80 |
| c [wt.-%] | ≥50 | ≥50 | ≥50 | ≥50 | ≥50 | ≥50 | ≥50 | ≥50 | ≥50 | ≥50 |
| | A²¹ | A²² | A²³ | A²⁴ | A²⁵ | A²⁶ | A²⁷ | A²⁸ | A²⁹ | A³⁰ |
| T [° C.] | ≤55 | ≤50 | ≤45 | ≤40 | ≤37 | ≤55 | ≤50 | ≤45 | ≤40 | ≤37 |
| p [mbar] | ≤500 | ≤500 | ≤500 | ≤500 | ≤500 | ≤200 | ≤200 | ≤200 | ≤200 | ≤200 |
| c [wt.-%] | ≥55 | ≥55 | ≥55 | ≥55 | ≥55 | ≥55 | ≥55 | ≥55 | ≥55 | ≥55 |
| | A³¹ | A³² | A³³ | A³⁴ | A³⁵ | A³⁶ | A³⁷ | A³⁸ | A³⁹ | A⁴⁰ |
| T [° C.] | ≤55 | ≤50 | ≤45 | ≤40 | ≤37 | ≤55 | ≤50 | ≤45 | ≤40 | ≤37 |
| p [mbar] | ≤100 | ≤100 | ≤100 | ≤100 | ≤100 | ≤80 | ≤80 | ≤80 | ≤80 | ≤80 |
| c [wt.-%] | ≥55 | ≥55 | ≥55 | ≥55 | ≥55 | ≥55 | ≥55 | ≥55 | ≥55 | ≥55 |
| | A⁴¹ | A⁴² | A⁴³ | A⁴⁴ | A⁴⁵ | A⁴⁶ | A⁴⁷ | A⁴⁸ | A⁴⁹ | A⁵⁰ |
| T [° C.] | ≤55 | ≤50 | ≤45 | ≤40 | ≤37 | ≤55 | ≤50 | ≤45 | ≤40 | ≤37 |
| p [mbar] | ≤500 | ≤500 | ≤500 | ≤500 | ≤500 | ≤200 | ≤200 | ≤200 | ≤200 | ≤200 |
| c [wt.-%] | ≥60 | ≥60 | ≥60 | ≥60 | ≥60 | ≥60 | ≥60 | ≥60 | ≥60 | ≥60 |
| | A⁵¹ | A⁵² | A⁵³ | A⁵⁴ | A⁵⁵ | A⁵⁶ | A⁵⁷ | A⁵⁸ | A⁵⁹ | A⁶⁰ |
| T [° C.] | ≤55 | ≤50 | ≤45 | ≤40 | ≤37 | ≤55 | ≤50 | ≤45 | ≤40 | ≤37 |
| p [mbar] | ≤100 | ≤100 | ≤100 | ≤100 | ≤100 | ≤80 | ≤80 | ≤80 | ≤80 | ≤80 |
| c [wt.-%] | ≥60 | ≥60 | ≥60 | ≥60 | ≥60 | ≥60 | ≥60 | ≥60 | ≥60 | ≥60 |
| | A⁶¹ | A⁶² | A⁶³ | A⁶⁴ | A⁶⁵ | A⁶⁶ | A⁶⁷ | A⁶⁸ | A⁶⁹ | A⁷⁰ |
| T [° C.] | ≤55 | ≤50 | ≤45 | ≤40 | ≤37 | ≤55 | ≤50 | ≤45 | ≤40 | ≤37 |
| p [mbar] | ≤500 | ≤500 | ≤500 | ≤500 | ≤500 | ≤200 | ≤200 | ≤200 | ≤200 | ≤200 |
| c [wt.-%] | ≥65 | ≥65 | ≥65 | ≥65 | ≥65 | ≥65 | ≥65 | ≥65 | ≥65 | ≥65 |
| | A⁷¹ | A⁷² | A⁷³ | A⁷⁴ | A⁷⁵ | A⁷⁶ | A⁷⁷ | A⁷⁸ | A⁷⁹ | A⁸⁰ |
| T [° C.] | ≤55 | ≤50 | ≤45 | ≤40 | ≤37 | ≤55 | ≤50 | ≤45 | ≤40 | ≤37 |
| p [mbar] | ≤100 | ≤100 | ≤100 | ≤100 | ≤100 | ≤80 | ≤80 | ≤80 | ≤80 | ≤80 |
| c [wt.-%] | ≥65 | ≥65 | ≥65 | ≥65 | ≥65 | ≥65 | ≥65 | ≥65 | ≥65 | ≥65 |

Preferably, the aqueous solution at the temperature of the solution has a viscosity of at most 2000 mPa·s, or at most 1950 mPa·s, or at most 1900 mPa·s, or at most 1850 mPa·s, or at most 1800 mPa·s, or at most 1750 mPa·s, or at most 1700 mPa·s, or at most 1650 mPa·s, or at most 1600 mPa·s, or at most 1550 mPa·s, or at most 1500 mPa·s, in each case measured by means of a rotary viscosimeter at a speed of 100 rpm.

A second aspect of the invention relates to an allulose syrup containing allulose at a product concentration of more than 70 wt.-%, relative to the total weight of the allulose syrup, wherein the allulose syrup in the CIELAB color space has an

(i) L* value greater than 94.60; and/or
(ii) a* value greater than −4.70; and/or
(iii) b* value lower than 21.70.

In preferred embodiments, the L* value of the allulose syrup obtained in step (b) is at least 94.70, or at least 94.80, or at least 94.90, or at least 95.00, or at least 95.10, or at least 95.20, or at least 95.30, or at least 95.40, or at least 95.50, or at least 95.60, or at least 95.70, or at least 95.80, or at least 95.90, or at least 96.00, or at least 96.10, or at least 96.20, or at least 96.30, or at least 96.40, or at least 96.50.

Preferably, the L* value of the allulose syrup obtained in step (a) is within the range of 96.75±2.00, more preferably 96.75±1.80, still more preferably 96.75±1.60, yet more preferably 96.75±1.40, even more preferably 96.75±1.20, most preferably 96.75±1.00, and in particular 96.75±0.80.

Preferably, the L* value of the allulose syrup obtained in step (b) relatively deviates from the L* value of the aqueous solution provided in step (a) by not more than ±2.50 units, more preferably not more than ±2.00 units, still more preferably by not more than ±1.50 units, yet more preferably by not more than ±1.00 units, even more preferably by not more than ±0.50 units, most preferably by not more than ±0.40 units, and in particular by not more than ±0.30 units.

Preferably, the L* value of the allulose syrup obtained in step (b) relatively deviates from the L* value of the starting material provided in step (a-1) by not more than ±2.50 units, more preferably not more than ±2.00 units, still more preferably by not more than ±1.50 units, yet more preferably by not more than ±1.00 units, even more preferably by not more than ±0.50 units, most preferably by not more than ±0.40 units, and in particular by not more than ±0.30 units.

In preferred embodiments, the a* value of the allulose syrup obtained in step (b) is at least −4.50, or at least −4.00, or at least −3.50, or at least −3.00, or at least −2.50, or at least −2.00, or at least −1.90, or at least −1.80, or at least −1.70, or at least −1.60, or at least −1.50, or at least −1.45, or at least −1.40, or at least −1.35, or at least −1.30, or at least −1.25.

Preferably, the a* value of the allulose syrup obtained in step (b) is within the range of −1.13±4.00, more preferably −1.13±3.50, still more preferably −1.13±3.00, yet more preferably −1.13±2.50, even more preferably −1.13±2.00, most preferably −1.13±1.50, and in particular −1.13±1.00. Preferably, the a* value of the allulose syrup obtained in step (b) is within the range of −1.13±0.90, more preferably −1.13±0.80, still more preferably −1.13±0.70, yet more preferably −1.13±0.60, even more preferably −1.13±0.40, most preferably −1.13±0.30, and in particular −1.13±0.20.

Preferably, the a* value of the allulose syrup obtained in step (b) relatively deviates from the a* value of the aqueous solution provided in step (a) by not more than ±10.00 units, more preferably not more than ±8.00 units, still more preferably by not more than ±6.00 units, yet more preferably by not more than ±4.00 units, even more preferably by not more than ±2.00 units, most preferably by not more than ±1.00 units, and in particular by not more than ±0.50 units.

Preferably, the a* value of the allulose syrup obtained in step (b) relatively deviates from the a* value of the starting material provided in step (a-1) by not more than ±10.00 units, more preferably not more than ±8.00 units, still more preferably by not more than ±6.00 units, yet more preferably by not more than ±4.00 units, even more preferably by not more than ±2.00 units, most preferably by not more than ±1.00 units, and in particular by not more than ±0.50 units.

In preferred embodiments, the b* of the allulose syrup obtained in step (b) value is at most 21.00, or at most 19.00, or at most 18.00, or at most 17.00, or at most 16.00, or at most 15.00, or at most 14.00, or at most 13.00, or at most 12.00, or at most 11.00, or at most 10.00, or at most 9.00, or at most 8.00, or at most 7.00, or at most 6.00, or at most 5.00, or at most 4.50, or at most 4.40, or at most 4.30, or at most 4.20, or at most 4.10, or at most 4.00, or at most 3.90, or at most 3.80, or at most 3.70, or at most 3.60, or at most 3.50.

Preferably, the b* value of the allulose syrup obtained in step (b) is within the range of −2.90±10.00, more preferably −2.90±9.00, still more preferably −2.90±8.00, yet more preferably −2.90±7.00, even more preferably −2.90±6.00, most preferably −2.90±5.00, and in particular −2.90±4.00. Preferably, the b* value of the allulose syrup obtained in step (b) is within the range of −2.90±3.50, more preferably −2.90±3.00, still more preferably −2.90±2.50, yet more preferably −2.90±2.00, even more preferably −2.90±1.80, most preferably −2.90±1.60, and in particular −2.90±1.40.

Preferably, the b* value of the allulose syrup obtained in step (b) relatively deviates from the b* value of the aqueous solution provided in step (a) by not more than ±3.00 units, more preferably not more than ±2.50 units, still more preferably by not more than ±2.00 units, yet more preferably by not more than ±1.50 units, even more preferably by not more than ±1.00 units, most preferably by not more than ±0.50 units, and in particular by not more than ±0.20 units.

Preferably, the b* value of the allulose syrup obtained in step (b) relatively deviates from the b* value of the starting material provided in step (a-1) by not more than ±3.00 units, more preferably not more than ±2.50 units, still more preferably by not more than ±2.00 units, yet more preferably by not more than ±1.50 units, even more preferably by not more than ±1.00 units, most preferably by not more than ±0.50 units, and in particular by not more than ±0.20 units.

Preferably, the color distance ΔE calculated from the data of the CIELAB color space in accordance with DIN EN ISO/CIE 11664-4:2020-03 (ΔE*_{ab}=[ΔL*)²+(Δa*)²+(Δb*)²]^{1/2}) of the allulose syrup obtained in step (b) and of the aqueous solution provided in step (a) is at most 15, more preferably at most 12, still more preferably at most 9, yet more preferably at most 7, even more preferably at most 5, most preferably at most 2, and in particular at most 1.5.

Preferably, the color distance ΔE calculated from the data of the CIELAB color space in accordance with DIN EN ISO/CIE 11664-4:2020-03 (ΔE*_{ab}=[(ΔL*)²+(Δa*)²+(Δb*)²]^{1/2}) of the allulose syrup obtained in step (b) and of the starting material provided in step (a-1) is at most 15, more preferably at most 12, still more preferably at most 9, yet more preferably at most 7, even more preferably at most 5, most preferably at most 2, and in particular at most 1.5.

Preferably, the allulose syrup obtained in step (b) has a density of at least 1.18 g·cm⁻³, more preferably at least 1.21 g·cm⁻³, still more preferably at least 1.24 g·cm⁻³, yet more preferably at least 1.27 g·cm⁻³, even more preferably at least 1.30 g·cm³, most preferably at least 1.33 g·cm⁻³, and in particular at least 1.36 g·cm³.

Preferably, the product concentration of the allulose syrup obtained in step (b) is at least 75 wt. %, preferably at least 77.5 wt.-%, more preferably at least 80 wt.-%, still more preferably at least 82.5 wt.-%, yet more preferably at least 85 wt.-%.

Preferably, the allulose syrup obtained in step (b) containing allulose at the product concentration is an aqueous solution which does not contain crystals.

Preferably, the allulose syrup obtained in step (b) essentially consists of (i) allulose, (ii) residual by-products obtained in the course of allulose synthesis and not removed by purification, (iii) residual starting materials not converted in the course of allulose synthesis and not removed by purification, and (iv) water. Preferably, the allulose syrup obtained in step (b) essentially contains no liquids (solvents) other than water.

Preferably, the allulose syrup obtained in step (b) has an allulose content of at least 90 wt.-%; preferably at least 95 wt.-%; more preferably at least 98 wt.-%; still more preferably at least 99 wt.-%; in each case relative to the total content of dry matter that is contained in the allulose syrup.

Preferably, the allulose syrup obtained in step (b) has a fructose content of at most 10 wt.-%; preferably at most 5.0 wt.-%; more preferably at most 2.5 wt.-%; in each case relative to the total content of dry matter that is contained in the allulose syrup.

Preferably, the allulose syrup obtained in step (b) has a content of components other than allulose (i.e. total impurities) of at most 10 wt.-%; preferably at most 5.0 wt.-%; more preferably at most 2.5 wt.-%; in each case relative to the total content of dry matter that is contained in the allulose syrup.

While it is contemplated that the allulose syrup may additionally contain undissolved material in suspension, preferably the allulose syrup is a pure solution.

Preferably, the allulose syrup according to the invention as described above is obtainable by or obtained by the process according to the invention as described above. Thus, another aspect of the invention relates to an allulose syrup obtainable by or obtained by the process according to the invention as described above.

The following examples further illustrate the invention but are not to be construed as limiting its scope.

### EXAMPLE 1

An allulose syrup having a dry substance content of 71.04 wt.-% was prepared and stored at different temperatures (22° C., 40° C., and 60° C., respectively). After 24 hours and after 168 hours, various properties were determined and compared to the respective properties of the allulose syrup prior to storage.

The results of dry substance and color measurements are compiled in the following table:

| | T [° C.] | 0 h | | 24 h | 168 h |
|---|---|---|---|---|---|
| dry substance | 22 | 71.04 | | 71.28 | 71.66 |
| (MA) [%] | 40 | | | 71.20 | 71.14 |
| | 60 | | | 71.23 | 70.86 |
| EBC color intensity | 22 | 0.28 | | 0.30 | 0.20 |
| [EBC] | 40 | | | 0.40 | 0.35 |
| | 60 | | | 0.56 | 3.25 |
| color in solution | 22 | 59 | | 54 | 58 |
| (MOPS) [IU] | 40 | | | 68 | 74 |
| | 60 | | | 80 | 327 |
| CIELAB | 22 | L* | 96.75 | 96.76 | 96.74 |
| color space | | a* | −1.13 | −1.12 | −1.06 |
| | | b* | 2.9 | 2.89 | 2.72 |
| | 40 | L* | 96.75 | 96.76 | 96.65 |
| | | a* | −1.13 | −1.15 | −1.26 |
| | | b* | 2.9 | 2.97 | 3.49 |
| | 60 | L* | 96.75 | 96.45 | 94.57 |
| | | a* | −1.13 | −1.48 | −4.73 |
| | | b* | 2.9 | 4.29 | 21.76 |
| absorbance of the syrup at 450 nm and subtracting | 22 | | 0.004 | 0.004 | 0.003 |
| the background at 600 nm and dividing the result | 40 | | 0.004 | 0.008 | 0.007 |
| by the path length of the cuvette | 60 | | 0.004 | 0.011 | 0.079 |

These results of dry substance and color measurements are further visualized in FIGS. 1 to 6.

The color distances ΔE calculated from the data of the CIELAB color space in accordance with DIN EN ISO/CIE 11664-4:2020-03 (ΔE*_{ab}=[ΔL*)²+(Δa*)²+(Δb*)²]^{1/2}) are compiled in the following table:

| ΔE | distance | value |
|---|---|---|
| 22° C. | 0 h −> 24 h | 0.02 |
| | 24 h −> 168 h | 0.18 |
| | 0 h −> 168 h | 0.19 |
| 40° C. | 0 h −> 24 h | 0.07 |
| | 24 h −> 168 h | 0.54 |
| | 0 h −> 168 h | 0.61 |
| 60° C. | 0 h −> 24 h | 1.46 |
| | 24 h −> 168 h | 17.87 |
| | 0 h −> 168 h | 19.32 |

The results of an organoleptic assessment by a trained panel (n=3) are compiled in the following table:

| | T [° C.] | 0 h | 24 h | 168 h |
|---|---|---|---|---|
| Appearance/ | 22 | slightly yellowish, | unchanged | unchanged |
| Color | | visible flow marks | (cf. sample at 0 h) | (cf. sample at 0 h and sample |
| | | (occurrence | | after 24 h at 22° C.) |
| | 40 | typical) viscous, | nearly identical in color tone | nearly identical in color tone |
| | | clear, | and color intensity like sample | and color intensity like sample |
| | | | at 0 h, clear | at 0 h and sample after 24 h at |
| | | | | 40° C. (minimal nuance |
| | | | | yellower), clear |
| | 60 | | slightly more intense yellowish, | yellow, clear |
| | | | clear, distinguishable from | |
| | | | sample after 24 h at 22° C. and | |
| | | | sample after 24 h at 40° C. | |
| Smell | 22 | very weakly | unchanged | weakly like cotton candy |
| | | caramel-like | (cf. sample at 0 h) | |
| | 40 | | slightly more intense caramel- | weakly like cotton candy |
| | | | like, slightly fruity, slightly | |
| | | | butter-like | |
| | 60 | | slightly more intense caramel- | slightly caramel-like but |
| | | | like, slightly fruity, slightly | significantly lower intensity |
| | | | butter-like, comparable to sample | than sample after 24 h at 60° C. |
| | | | after 24 h at 40° C. | |
| Taste/ | 22 | sweet, cream | unchanged | sweet, slightly caramel-like, |
| Flavor | | caramel-like, | (cf. sample at 0 h) | retronasal |
| | 40 | candy-like | caramel taste somewhat | sweet, cream caramel-like, |
| | | | changed in comparison to | candy-like soft |
| | | | sample at 0 h, slightly less | |
| | | | cream caramel-like, slightly | |
| | | | more butter-like, slightly | |
| | | | fruitier, slightly rougher | |
| | 60 | | weaker caramel-like in | sweet, caramel-like, |
| | | | comparison to sample at 0 h | roasty/mildewed, slightly |
| | | | | alt, butter-like/fatty, changing |
| | | | | mouthfeel |

consistency/texture: in each case at each time: syrup-like, viscous

### EXAMPLE 2

The following devices were used:
rotatory evaporator Rotavapor R-220 SE, Co. Büchi;
refractometer Pure S, Co. Schmidt und Haensch;
Heidolph Hei-Torque 200, Co. Heidolph Instruments;
allulose syrup L3121034, Co. Savanna Ingredients; and
thermometer G1720-GE, Co. Greisinger.

Refractometric index (RI)/refractive index (Brix) was determined at 20° C. from the samples using a refractometer (Pure S, Co. Schmidt und Haensch). The dry substance content (ds) of the samples was determined according to Eq. 1:

ds=RI*592.564−787.316 Eq. 1

An allulose syrup having a dry substance content (ds) of 71.35 wt.-% was diluted to a dry substance content of 51.16 wt.-%. The CIELAB color space was measured before and after dilution. Additionally, the CIELAB color space of the centrifuge effluent of an allulose syrup after crystallization was measured:

| | | | CIELAB color space | | |
|---|---|---|---|---|---|
| Probe | | ds [wt.-%] | L* | a* | b* |
| 1 | crude | 71.35 | 96.45 | −1.08 | 3.48 |
| 2 | starting | 51.16 | 96.18 | −0.72 | 2.25 |
| 7 | centrifuge effluent | n.d. | 90.64 | −4.76 | 48.12 |

The diluted allulose syrup (ds 51.16 wt.-%) was divided into separate fractions and each fraction was evaporated at different temperatures and different pressures to a dry substance content of about 80 wt.-%. In Examples 2-1 and 2-2, temperature and pressure were constant over the whole evaporation (single-stage-evaporation). In Example 2-3, evaporation was carried out in two stages beginning at a higher temperature (two-stage-evaporation).

Before and after evaporation, the CIELAB color space was measured. The color distances ΔE are calculated after evaporation relative to the starting allulose syrup (ds 51.16 wt.-%) from the data of the CIELAB color space in accordance with DIN EN ISO/CIE 11664-4:2020-03 (ΔE*_{ab}=[(ΔL*)²+(Δa*)²+(Δb*)²]^{1/2}).

The evaporation conditions and the results of dry substance and color measurements are compiled in the following table:

| | dry substance | | | temperature | | | | CIELAB color space | | | |
|---|---|---|---|---|---|---|---|---|---|---|---|
| Ex. | start | | end | bath | solution | pressure | | L* | a* | b* | ΔE |
| 2-1 3 | 51.16% | | ~80% | mild 55° C. | 48° C. | 70 | mbar | 96.45 | −1.32 | 4.12 | 1.98 |
| 2-2 6 | 51.16% | | ~80% | harsh 80° C. | 70° C. | 210 | mbar | 96.36 | −1.52 | 4.94 | 2.81 |
| 2-3 4.5 | 1. | 51.16% | ~65% | harsh 80° C. | 68° C. | 210 | mbar | 96.58 | −1.03 | 3.42 | 1.27 |
| | 2. | ~65% | ~80% | mild 55° C. | 50° C. | 70 | mbar | 96.57 | −1.3 | 4.18 | 2.05 |

As demonstrated by the above comparative experimental data, one-stage-evaporation under harsh temperature conditions (70° C., ds 51.16%->˜80%) results in the most significant color distance (Example 2-2; ΔE 2.81). In contrast, color distance upon two-stage-evaporation beginning at harsh temperature conditions (68° C., ds 51.16%->˜65%) followed by mild temperature conditions (50° C., ds ˜65%->˜80%) (Example 2-3; ΔE 2.05) is comparable to color distance upon one-stage-evaporation under mild temperature conditions (48° C., ds 51.16%->˜80%) (Example 2-1).

This effect cannot be merely based upon different exposure times of the material at different temperatures because lower temperatures were compensated by stronger vacuum (lower pressure) such that overall evaporation times were similar.

| Probe | | | Brix | RI | ds |
|---|---|---|---|---|---|
| 1 | crude | | 67.51 | 1.45929 | 71.35 |
| 2 | starting | | 47.57 | 1.41501 | 51.16 |
| 3 | 2-1 | | 75.24 | 1.47878 | 80.23 |
| 6 | 2-2 | | 79.50 | 1.47690 | 74.62 |
| 4 | 2-3 | 1. | 64.00 | 1.45114 | 67.58 |
| 5 | | 2. | 74.26 | 1.47600 | 79.08 |

It can be concluded from the above comparative experimental data as long as the dry substance content of allulose syrups is not more than 70 wt.-%, evaporation can be performed at comparatively high temperatures, e.g. at 80° C., without causing significant color distance relative to the starting material. Once a dry substance content of 70 wt.-% is reached, however, evaporation should not proceed under such harsh conditions but should be continued at lower temperatures, e.g. at 55° C., in order to prevent significant color distance formation, i.e. discoloration.

What is claimed is:

1. A process for the preparation of an allulose syrup containing allulose at a product concentration of more than 70 wt.-%, relative to the total weight of the allulose syrup, the process comprising the steps of
(a) providing an aqueous solution containing allulose at an educt concentration of at most 70 wt.-%, relative to the total weight of the solution; and
(b) evaporating water at a temperature of the solution of less than 60° C. and under reduced pressure thereby increasing the concentration of allulose in the aqueous solution starting from the educt concentration until the product concentration is reached.

2. The process according to claim 1, wherein the temperature of the solution is at most 55° C., preferably at most 50° C., more preferably at most 45° C., still more preferably at most 40° C., yet more preferably at most 37° C.

3. (canceled)

4. The process of claim 1, wherein the temperature of the solution is at least 36° C., preferably at least 38° C., more preferably at least 40° C., still more preferably at least 42° C., yet more preferably at least 44° C., even more preferably at least 46° C., most preferably at least 48° C., and in particular at least 50° C.

5. (canceled)

6. The process of claim 1, wherein step (b) is carried out at a temperature of at least 36° C., preferably at least 38° C., more preferably at least 40° C., still more preferably at least 42° C., yet more preferably at least 44° C., even more preferably at least 46° C., most preferably at least 48° C., and in particular at least 50° C.

7. (canceled)

8. The of claim 1, wherein the pressure is at most 500 mbar, preferably at most 200 mbar, more preferably at most 100 mbar, still more preferably at most 80 mbar;
optionally wherein the pressure is at least 50 mbar, preferably at least 70 mbar, more preferably at least 90 mbar, still more preferably at least 110 mbar, yet more preferably at least 130 mbar, even more preferably at least 150 mbar, most preferably at least 170 mbar, and in particular at least 190 mbar.

9. (canceled)

10. The process of claim 1,
wherein step (b) is carried out at a pressure of at most 499 mbar, preferably at most 399 mbar, more preferably at most 350 mbar, still more preferably at most 300 mbar, yet more preferably at most 250 mbar, even more preferably at most 200 mbar, most preferably at most 150 mbar, and in particular at most 100 mbar; and/or
wherein step (b) is carried out at a pressure of at least 50 mbar, preferably at least 70 mbar, more preferably at least 90 mbar, still more preferably at least 110 mbar, yet more preferably at least 130 mbar, even more preferably at least 150 mbar, most preferably at least 170 mbar, and in particular at least 190 mbar.

11. (canceled)

12. (canceled)

13. The process of claim 1, wherein the aqueous solution provided in step (a) contains allulose at an educt concentration of at least 50 wt.-%, preferably at least 52.5 wt.-%, more preferably at least 55 wt.-%, still more preferably at least 57.5, yet more preferably at least 60 wt.-%, even more preferably at least 62.5 wt.-%, most preferably at least 65 wt.-%, relative to the total weight of the solution.

14. The process of claim 1, wherein the aqueous solution provided in step (a) contains allulose at an educt concentration of at most 67.5 wt.-%, preferably at most 65 wt.-%, more preferably at most 62.5 wt.-%, still more preferably at most 60 wt.-%, relative to the total weight of the solution.

15. The process of claim 1, wherein the aqueous solution provided in step (a) in the CIELAB color space has an
(i) L* value greater than 94.60; and/or
(ii) a* value greater than −4.70; and/or
(iii) b* value lower than 21.70.

16. (canceled)

17. The process of claim 1, wherein the color distance ΔE calculated from the data of the CIELAB color space in accordance with DIN EN ISO/CIE 11664-4:2020-03 (ΔE*_{ab}=[(ΔL*)²+(Δa*)²+(Δb*)²]^{1/2}) of the allulose syrup obtained in step (b) and of the aqueous solution provided in step (a) is at most 15, more preferably at most 12, still more preferably at most 9, yet more preferably at most 7, even more preferably at most 5, most preferably at most 2, and in particular at most 1.5.

18. The process of claim 1, wherein the product concentration is at least 75 wt.-%, preferably at least 77.5 wt.-%, more preferably at least 80 wt.-%, still more preferably at least 82.5 wt.-%, yet more preferably at least 85 wt.-%.

19. (canceled)

20. The process of claim 1, wherein step (a) comprises a preconcentration step, which comprises the sub-steps of
(a-1) providing a starting material containing allulose at a starting concentration of at least 25 wt.-%; and
(a-2) evaporating water at a temperature of the starting material of more than 35° C. and under reduced pressure thereby increasing the concentration of allulose in the starting material starting from the starting concentration until the educt concentration is reached and thereby providing the aqueous solution containing allulose at the educt concentration.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. The process of claim 20, wherein the starting material provided in sub-step (a-1) contains allulose at a starting concentration of at least 30 wt.-%, preferably at least 35 wt.-%, more preferably at least 40 wt.-%, still more preferably at least 42 wt.-%, yet more preferably at least 44 wt.-%, even more preferably at least 46 wt.-%, most preferably at least 48 wt.-%, and in particular at least 50 wt.-%, relative to the total weight of the starting material; and/or
wherein the starting material provided in sub-step (a-1) contains allulose at a starting concentration of at most 69 wt.-%, preferably at most 67 wt.-%, more preferably at most 64 wt.-%, still more preferably at most 62 wt.-%, yet more preferably at most 59 wt.-%, even more preferably at most 57 wt.-%, most preferably at most 54 wt.-%, and in particular at most 52 wt.-%, relative to the total weight of the starting material.

33. (canceled)

34. (canceled)

35. The process of claim 20, the allulose content of the starting material at the starting concentration is less than the allulose content at the aqueous solution at the educt concentration.

36. (canceled)

37. (canceled)

38. (canceled)

39. The process of claim 20, which comprises the steps of
(a-1) providing a starting material containing allulose at a starting concentration of at least 50 wt.-%; and
(a-2) evaporating water at a temperature of the starting material of at least 62° C. and at a pressure of at most 210 mbar thereby increasing the concentration of allulose in the starting material starting from the starting concentration until the educt concentration is reached and thereby providing the aqueous solution containing allulose at the educt concentration; and
(b) evaporating water at a temperature of the solution within the range of from 46 to 54° C. and at a pressure of at most 70 mbar thereby increasing the concentration of allulose in the aqueous solution starting from the educt concentration until the product concentration is reached.

40. The process of claim 20, wherein the conditions of the preconcentration step (a) essentially correspond or are identical to the conditions of the evaporation step (b);
optionally wherein the temperature of the starting material in the preconcentration step (a) relatively deviates from the temperature of the solution in the evaporation step (b) by not more than 5%, preferably not more than 2%;
optionally wherein the pressure in the preconcentration step (a) relatively deviates from the pressure in the evaporation step (b) by not more than 5%, preferably not more than 2%.

41. (canceled)

42. (canceled)

43. (canceled)

44. The process of claim 1, wherein the conditions of the preconcentration step (a) differ from the conditions of the evaporation step (b) in
(i) the temperature of the starting material and the solution, respectively;
(ii) the pressure; or
(iii) the temperature of the starting material and the solution, respectively and the pressure.

45. (canceled)

46. The process of claim 1, wherein the preconcentration step (a) and the evaporation step (b) are performed under essentially the same pressure;
optionally wherein the pressure in the preconcentration step (a) relatively deviates from the pressure in the evaporation step (b) by not more than 5%, preferably not more than 2%.

47. (canceled)

48. (canceled)

49. The process of claim 46, wherein the relative difference of the temperature of the starting material in the preconcentration step (a) and the temperature of the solution in the evaporation step (b) is at least 5° C., or at least 10° C., or at least 15° C., or at least 20° C., or at least 25° C., or at least 30° C., or at least 35° C., or at least 40° C.

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. An allulose syrup containing allulose at a product concentration of more than 70 wt.-%, relative to the total weight of the allulose syrup, wherein the allulose syrup in the CIELAB color space has an
(i) L* value greater than 94.60; and/or
(ii) a* value greater than −4.70; and/or
(iii) b* value lower than 21.70.

79. (canceled)

80. (canceled)
